(19) United States
(12) Patent Application Publication
(10) Pub. No.: US 2022/0241533 A1
(43) Pub. Date: Aug. 4, 2022

(54) OXYGEN MASK WITH CARBON DIOXIDE MONITOR

(71) Applicants: LOGAN HENRY FAIRCHILD, Brooklyn, NY (US); Trevor Gibbs, Naperville, IL (US); Mohamad Shafik Hashim, Saddle River, NJ (US)

(72) Inventors: LOGAN HENRY FAIRCHILD, Brooklyn, NY (US); Trevor Gibbs, Naperville, IL (US); Mohamad Shafik Hashim, Saddle River, NJ (US)

(21) Appl. No.: 17/588,330

(22) Filed: Jan. 30, 2022

Related U.S. Application Data
(60) Provisional application No. 63/144,423, filed on Feb. 1, 2021.

(51) Int. Cl. A61M 16/06

(52) U.S. Cl. CPC A61M 16/06; A61M 2205/18; A61M 2230/43

(57) ABSTRACT

An oxygen mask that incorporates chemical carbon dioxide (EtCO2) detectors to allow for the monitoring of patient respiration/ventilation without the use of clinical signs or electronic computer technology by providing a visual indication (through color change of the EtCO2 detectors) of the presence or absence of carbon dioxide.

### CROSS REFERENCE TO RELATED APPLICATIONS

The present application is a non-provisional of, and claims benefit of priority from, U.S. Provisional Patent Application No. 63/144,423, filed Feb. 1, 2021, the entirety of which is expressly incorporated herein by reference.

### FIELD OF THE INVENTION

The present invention relates to the field of face masks, and more specifically to masks having a visual capnometer display.

### BACKGROUND OF INVENTION

Each reference cited herein is expressly incorporated by reference in its entirety, for all purposes.

There are certain fields of medicine and patient care settings where continuous and continual monitoring of vital signs is necessary. These instances include but are not limited to emergency medicine, procedure-based medicine, anesthesiology, radiology, and in patient medicine. Frequently, clinicians look to electronically monitored vital signs or clinical signs to determine if a patient is in stable condition or if further action is needed to provide optimal care to a patient.

Currently, monitoring the ventilation/respiration of certain spontaneously breathing patient populations, such as unconscious patients during transport, is achieved by subjective observation, including chest movement, or by complicated electronic computerized machines. Regular monitoring of ventilation/respiration is critical practice in hospitals, ambulatory surgical centers, medical and dental offices, EMS vehicles, and police and fire response vehicles.

Visual monitoring of respiration is difficult and time consuming, and reliance on non-contact visual observation of respiration is fraught with error and subject to inter-healthcare practitioner variation.

Supplemental oxygen masks are ubiquitous in medicine. They are used by EMS services, inpatient, outpatient, operating room, intensive care unit, emergency room, gastrointestinal procedure suites, just to name a few. These masks supply additional oxygen. However, the supplemental oxygen is only able to help patients if the oxygen is breathed in and out of the lungs. This is called ventilation.

In medicine, ventilation is defined as the movement of gases in and out of the lungs. There are many situations where the patient does not, or cannot, self-ventilate air and oxygen.

Determining respiratory rate and ventilation are key vital signs in many fields and situations in medicine. For example, a patient who is undergoing or recovering from anesthesia/sedation. A patient under anesthesia or recovering from anesthesia may appear to be breathing and their vitals may be stable, but the patient might not be ventilating properly and will become very quickly very unstable. In patients with altered levels of consciousness (i.e., patients undergoing sedation, anesthesia, critically ill patients, and patients with altered mental status due to a medical or psychological ailments), a lapse in the monitoring of patient ventilation/respiration can have catastrophic repercussions which include but are not limited to cardiac arrest, anoxic brain injury, and death.

Patients who are compromised either by their medical comorbidities or due to sedation are given a supplemental oxygen mask to help the patient maintain normal blood oxygen levels. It may be necessary to confirm that this oxygen has been breathed into the lungs and transferred to the body, especially in a patient with impaired communication or consciousness. These supplemental oxygen masks make additional oxygen available, but do not breathe for the patient and do not confirm that the patient is actually ventilating their lungs. It is possible, and often happens, that a patient has a mask applied, but they are not making respiratory efforts and therefore the oxygen they need does not enter their bodies. Further complicating the matter is the fact that patients may in fact be making a respiratory effort, but not actually move any air. So a caregiver could see the patient look like they are breathing and be making respiratory effort, but not actually be ventilating their lungs. A common example of this is obstructive sleep apnea (OSA). In OSA, the anatomy of the patient causes the patient's airway to close and not allow air to move. And similar to OSA, patients with an altered mental status can obstruct their airways, without having a clinical diagnosis of OSA.

This airway obstruction is a common risk in patients compromised by medical conditions or sedation. A patient who is not ventilating, is not breathing. This is a critical situation and can quickly lead to cardiac arrest, anoxic brain injury, and death unless without prompt recognition and intervention.

Being able to identify that the patient is breathing is important and can be difficult. Many clinical providers are taught to count respiratory rate, via observed physical/clinical signs. While that measures the patient's respiratory effort, it does not identify the presence airway obstruction despite respiratory effort. Many clinical providers are not aware how to identify signs of obstructive apnea, and inter-patient and inter-practitioner variability exist.

During normal metabolism in the body, carbon dioxide (CO₂) is produced as a byproduct. This CO₂ is transported to the lungs to be breathed out. In a normal breath, oxygen is breathed in and CO₂ is breathed out. The ability to measure CO₂ near the mouth and nose confirms that air is being breathed in and out of the body. If there is no breathing, no ventilation, or if there is obstruction, there will not be any CO₂ detected around the face.

There are two current methods of detecting CO₂ during respiration. First, there is an expensive device called a capnometer that is used to measure the carbon dioxide during expiration for the quantification of respiration/ventilation/respiratory rate in the spontaneously breathing patient. This device needs an electric power source and special training to be able to use it effectively. Additionally, there is setup time to use it. Further barriers to using the device are the difficulty transporting the machine with a patient who is being transported in the hospital, which often times leads to practitioners forgoing its use during transport or when time efficiency is of importance. This happens quite often as patients need tests and procedures. Further, many hospital transporters are not trained to evaluate the quality of patient respiration and ventilation and even skilled healthcare practitioners can be fooled by inadequate respiratory efforts as well as succumbing to distraction while monitoring patients visually. The other type is a Chemical carbon dioxide detector which is a well established technology already used in the medical setting. For example, the Nellcor™ Adult/Pediatric Colorimetric CO₂ Detector, or Nellcor Easy Cap II CO₂ Detector, which attach directly to an endotracheal tube (ETT), to help clinicians verify proper ETT placement. See, www.medtronic.com/covidien/en-us/products/intubation/nellcor-adult-pediatric-colorimetric-co2-detector.html.

There are many situations where electronic/computerized detection of ventilation/respiration are either not possible or not available (i.e., rural/underserved healthcare communities, some urgent care centers, office based medical practices, emergency medical services vehicles, patient transport within large hospitals, outpatient/ambulatory surgery centers, and certain ill-prepared areas of large hospitals, amongst others).

There is need for a durable, low cost, and immediately available method to detect that the patient is ventilating. A mask with such capabilities could be kept in public settings along with AED (automated external defibrillators) to aid the layperson attempting to determine if the patient is breathing. They could also be kept in all parts of the hospital so they are immediately available with no setup. This mask could also be a huge benefit to areas that are unable to purchase the expensive computerized CO₂ monitors/detectors such as the underserved areas previously mentioned.

Chromogenic indicators for CO₂ may operate by sensing changes in pH due to formation of carbonic acid. They may employ litmus paper, or Bromothymol blue (also known as bromothymol sulfone phthalein and BTB.

A pH indicator is a halochromic chemical compound added in small amounts to a solution so the pH (acidity or basicity) of the solution can be determined visually. Hence, a pH indicator is a chemical detector for hydronium ions (H₃O⁺) or hydrogen ions (H⁺) in the Arrhenius model. Normally, the indicator causes the color of the solution to change depending on the pH. Indicators can also show change in other physical properties; for example, olfactory indicators show change in their odor. The pH value of a neutral solution is 7.0 at 25° C. (standard laboratory conditions). Solutions with a pH value below 7.0 are considered acidic and solutions with pH value above 7.0 are basic. Since most naturally occurring organic compounds are weak electrolytes, such as carboxylic acids and amines, pH indicators find many applications in biology and analytical chemistry. Moreover, pH indicators form one of the three main types of indicator compounds used in chemical analysis. For the quantitative analysis of metal cations, the use of complexometric indicators is preferred, whereas the third compound class, the redox indicators, are used in redox titrations (titrations involving one or more redox reactions as the basis of chemical analysis). In and of themselves, pH indicators are usually weak acids or weak bases. See en.wikipedia.org/wiki/PH_indicator.

Usually, the color change is not instantaneous at the pKa or pKb value, but a pH range exists where a mixture of colors is present. This pH range varies between indicators, but as a rule of thumb, it falls between the pKa or pKb value plus or minus one. This assumes that solutions retain their color as long as at least 10% of the other species persists. For example, if the concentration of the conjugate base is 10 times greater than the concentration of the acid, their ratio is 10:1, and consequently the pH is pKa+1 or pKb+1. Conversely, if a 10-fold excess of the acid occurs with respect to the base, the ratio is 1:10 and the pH is pKa−1 or pKb−1.

For optimal accuracy, the color difference between the two species should be as clear as possible, and the narrower the pH range of the color change the better. In some indicators, such as phenolphthalein, one of the species is colorless, whereas in other indicators, such as methyl red, both species confer a color. While pH indicators work efficiently at their designated pH range, they are usually destroyed at the extreme ends of the pH scale due to undesired side reactions.

| | Low pH | Transition | Transition | High pH |
|---|---|---|---|---|
| Indicator | color | low end | high end | color |
| Gentian violet (Methyl | yellow | 0 | 2 | blue- |
| violet 10B) | | | | violet |
| Malachite green (first | yellow | 0 | 2 | green |
| transition) | | | | |
| Malachite green (second | green | 11.6 | 14 | colorless |
| transition) | | | | |
| Thymol blue (first | red | 1.2 | 2.8 | yellow |
| transition) | | | | |
| Thymol blue (second | yellow | 8 | 9.6 | blue |
| transition) | | | | |
| Methyl yellow | red | 2.9 | 4 | yellow |
| Methylene blue | colorless | 5 | 9 | dark blue |
| Bromophenol blue | yellow | 3 | 4.6 | blue |
| Congo red | blue- | 3 | 5 | red |
| | violet | | | |
| Methyl orange | red | 3.1 | 4.4 | yellow |
| Screened methyl orange | red | 0 | 3.2 | purple- |
| (first transition) | | | | grey |
| Screened methyl orange | purple- | 3.2 | 4.2 | green |
| (second transition) | grey | | | |
| Bromocresol green | yellow | 3.8 | 5.4 | blue |
| Methyl red | red | 4.4 | 6.2 | yellow |
| Methyl purple | purple | 4.8 | 5.4 | green |
| Azolitmin (litmus) | red | 4.5 | 8.3 | blue |
| Bromocresol purple | yellow | 5.2 | 6.8 | purple |
| Bromothymol blue (first | magenta | <0 | 6 | yellow |
| transition) | | | | |
| Bromothymol blue | yellow | 6 | 7.6 | blue |
| (second transition) | | | | |
| Phenol red | yellow | 6.4 | 8 | red |
| Neutral red | red | 6.8 | 8 | yellow |
| Naphtholphthalein | pale red | 7.3 | 8.7 | greenish- |
| | | | | blue |
| Cresol red | yellow | 7.2 | 8.8 | reddish- |
| | | | | purple |
| Cresolphthalein | colorless | 8.2 | 9.8 | purple |
| Phenolphthalein (first | orange- | <0 | 8.3 | colorless |
| transition) | red | | | |
| Phenolphthalein (second | colorless | 8.3 | 10 | purple- |
| transition) | | | | pink |
| Phenolphthalein (third | purple- | 12 | 13 | colorless |
| transition) | pink | | | |
| Thymolphthalein (first | red | <10 | 9.3 | colorless |
| transition) | | | | |
| Thymolphthalein | colorless | 9.3 | 10.5 | blue |
| (second transition) | | | | |
| Alizarine Yellow R | yellow | 10.2 | 12 | red |
| Indigo carmine | blue | 11.4 | 13 | yellow |

Jain H, Vargese C. “Use Of Disposable End Tidal Carbon Dioxide Detector Device For Checking Endotracheal Tube Placement”, Journal of Clinical and Diagnostic Research Jun. 3, 2007; 1:104-109. www.jcdr.net/back_issues.asp?issn=0973-709x&year=2007&month=June&volume=1&issue=3&page=104-109&id=65
Schwarzenbach, Gerold (1957). Complexometric Titrations. Translated by Irving, Harry (1st English ed.). London: Methuen & Co. pp. 29-46.
West, T. S. (1969). Complexometry with EDTA and related reagents (3rd ed.). Poole, UK: BDH Chemicals Ltd. pp. 14-82.
Zumdahl, Steven S. (2009). Chemical Principles (6th ed.). New York: Houghton Mifflin Company. pp. 319-324.
Smyrnios, N. A., Lenard, R., Rajan, S., Newman, M. S., Baker, S. P., Thakkar, N., . . . Irwin, R. S. (2015). Comparison of a Self-Inflating Bulb Syringe and a Colorimetric CO 2 Indicator With Capnography and Radiography to Detect the Misdirection of Naso/Orogastric Tubes Into the Airway of Critically Ill Adult Patients. Chest, 147(6), 1523-1529. doi:10.1378/chest.14-0663.
John Pisowicz, Scott DeBoer, Michelle Webb, “Airway, Breathing or Consequences: Use Your Tools and Trust the Technology”, www.jems.com/patient-care/airway-respiratory/airway-breathing-or-consequences-use-your-tools-and-trust-the-technology/
Brown, Melissa K., Danielle V. Lazarus, Sarah R. Gonzales, Wade D. Rich, Madeline J. Wozniak, Debra M. Poeltler, and Anup C. Katheria. “Resistance of colorimetric carbon dioxide detectors commonly utilized in neonates.” Respiratory care 61, no. 8 (2016): 1003-1007.
Puntervoll, S. A., E. Søreide, W. Jacewicz, and E. Bjelland. “Rapid detection of oesophageal intubation: take care when using colorimetric capnometry.” Acta anaesthesiologica scandinavica 46, no. 4 (2002): 455-457.
Keller, William Ryan, Jefrey Biehler, Marc Yves-Rene Linares, and Barbara M. Garcia-Pena. “False-positive colorimetric capnometry after ingestion of carbonated beverages.” Pediatric emergency care 25, no. 2 (2009): 69-73.
Erzincanli, Saadet, Ayten Zaybak, and Ayşe Güler “Investigation of the efficacy of colorimetric capnometry method used to verify the correct placement of the nasogastric tube.” Intensive and Critical Care Nursing 38 (2017): 46-52.
Chau, Janita P C, Suzanne H S Lo, David R. Thompson, Ritin Fernandez, and Rhonda Griffiths. “Use of end-tidal carbon dioxide detection to determine correct placement of nasogastric tube: a meta-analysis.” International journal of nursing studies 48, no. 4 (2011): 513-521.
Del Rey, Javier A. Gonzalez, Michael P. Poirier, and Gregg A. Digiulio. “Evaluation of an ambu-bag valve with a self-contained, colorimetric end-tidal CO2 system in the detection of airway mishaps: an animal trial.” Pediatric emergency care 16, no. 2 (2000): 121-123.
DeBoer, Scott, Michael Seaver, and Karen Arndt. “Verification of endotracheal tube placement: a comparison of confirmation techniques and devices.” J Emerg Nurs 29, no. 5 (2003): 444-450.
Zwerneman, Karen. “End-tidal carbon dioxide monitoring: a VITAL sign worth watching.” Critical Care Nursing Clinics 18, no. 2 (2006): 217-225.
www.mercurymed.com/wp-content/uploads/pom_nora_article.pdf
web.a.ebscohost.com/ehost/detail/detail?vid=0&sid=528541bc-af9f-40c0-8ddd-aa66415869e2%40sessionmgr4007&bdata=JnNpdGU9ZWhvc3QtbG12ZQ %3d %3d #AN=12994 5908&db=ccm
www.ingentaconnect.com/content/wk/ane/2019/00000131/00000001/art00079
www.sciencedirect.com/science/article/abs/pii/S1553725016300915
www.ingentaconnect.com/content/wk/ane/2017/00000124/00000004/art00032
www.sciencedirect.com/science/article/abs/pii/S0952818016304135
journals.1ww.com/co-anesthesiology/fulltext/2009/08000/The_risk_and_safety_of anesthesia_at_remote.9.aspx
pubs.asahq.org/anesthesiology/article/104/2/228/631/Injury-and-Liability-Associated-with-Monitored
pubs. asahq.org/anesthesiology/article/104/2/228/631/Injury-and-Liability-Associated-with-Monitored
onlinelibrary.wiley.com/doi/10.1111/pan.12342
www.ncbi.nlm.nih.gov/pmc/articles/PMC5470361/
US 20190167171, 20180335440, 20180328841, and 20160245830 discuss colorimetric capnometry.
DE 20120543 U1 discloses respirator mask having an optically responsive chemical component, including a CO₂ concentration indicator.
See, AU2001268349; AU2002301160; AU2002362752; AU2002364046; AU2002364046; AU2003232110; AU2003232110; AU2005201500; AU2005201500; AU2006318572; AU2006318572; AU2007201370; AU2007201370; AU2008257519; AU2008257519; AU2008257519; AU2016320638; AU2016320638; AU2018358349; AU2018360849; AU2019283967; AU2019283967; AU2021250976; AU3306799; AU4325999; AU750050; AU757615; BR102018073943; BR102018074807; BRPI0618896; CA1340050; CA2305189; CA2321253; CA2334408; CA2379188; CA2398949; CA2412485; CA2463374; CA2472098; CA2472098; CA2485631; CA2726597; CA2726604; CA2773048; CA2773048; CA2808379; CA2808379; CA2865876; CA2936566; CA2941845; CA2997896; CA3023591; CA3024430; CA3081341; CA3081342; CN100361716; CN100542475; CN101065049; CN101296721; CN101296721; CN101432036; CN101454041; CN101454041; CN101584211; CN101588832; CN101588832; CN101905056; CN101905056; CN101912655; CN101912656; CN101927049; CN101954137; CN101954137; CN101954138; CN101954138; CN101961516; CN101961516; CN101961519; CN101961519; CN101961521; CN101961522; CN101961523; CN102186525; CN102187343; CN102448530; CN102625720; CN103037925; CN103096981; CN103313747; CN103313747; CN103619390; CN105597207; CN107106083; CN108883249; CN109996492; CN112996429; CN1455690; CN209575457U; DE102007039004; DE69831016T2; DE69936767T2; DK2822564T3; EP0364567; EP0364567; EP1056498; EP1056498; EP1058570; EP1058570; EP1082056; EP1082056; EP1202667; EP1251773; EP1359960; EP1450888; EP1450888; EP1467791; EP1573450; EP1706025; EP1951348; EP1951348; EP2167178; EP2344035; EP2470065; EP2473220; EP2473220; EP2486850; EP2605698; EP2605698; EP2611485; EP2611485; EP2663353; EP2707068; EP2707068; EP2819577; EP2822563; EP2822564; EP2822564; EP2822565; EP2822566; EP2839777; EP2839777; EP2964302; EP3066978; EP3094238; EP3148622; EP3169388; EP3197464; EP3229664; EP3347096; EP3421042; EP3510924; EP3510924; EP3511043; EP3511043; EP3568062; EP3620194; EP3643229; EP3703562; EP3703660; ES2289815T3; ES2297940T3; ES2362085T3; ES2690398T3; ES2820285T3; ES2859563T3; FR2905275; FR3076462; FR3076463; FR3087343; IL234508; IN2008DN04611; IN201947032664; IN202121025400; JP2003505180; JP2004506457; JP2005000675; JP2005514122; JP2005516644; JP2005537041; JP2006507905; JP2007518480; JP2009508645; JP2010528723; JP2011005262; JP2011529709; JP2012104146; JP2013503719; JP2014518725; JP2015037579; JP2017512556; JP2018532710; JP2019126426; JP2019517835; JP2020513934; JP2020530908; JP2021020111; JP2021130715; JP3688994; JP3738278; JP3842212; JP4377226; JP4751338; JP5053506; JP5296892; JP5628669; JP5758898; JP6001029; JP6788503; JPH10504733; KR20180066088; MXPA00011835; MXPA04003443; MXPA04006417; NL1042108; TWI747981; U.S. Ser. No. 10/039,781; U.S. Ser. No. 10/058,269; U.S. Ser. No. 10/108,325; U.S. Ser. No. 10/112,024; U.S. Ser. No. 10/137,245; U.S. Ser. No. 10/137,265; U.S. Ser. No. 10/383,546; U.S. Ser. No. 10/410,750; U.S. Ser. No. 10/468,138; U.S. Ser. No. 10/535,433; U.S. Ser. No. 10/729,334; U.S. Ser. No. 10/857,319; U.S. Ser. No. 10/877,655; U.S. Ser. No. 10/925,500; U.S. Ser. No. 10/974,002; U.S. Ser. No. 11/213,225; U.S. Ser. No. 11/219,728; US20010029340; US20020017296; US20020017299; US20020017300; US20020029004; US20020138213; US20030023182; US20030028120; US20030065273; US20030065274; US20030065275; US20030079746; US20030105407; US20030130591; US20030145854; US20030214409; US20040230108; US20040260161; US20050010165; US20050010166; US20050112325; US20060093785; US20060149144; US20060155206; US20060235324; US20070027375; US20070095347; US20070113849; US20070129647; US20070191688; US20070191697; US20070213658; US20080021379; US20080295839; US20090314290; US20100093800; US20100099999; US20100174161; US20100234705; US20110094518; US20110208539; US20110257550; US20120078069; US20120165623; US20120302845; US20120302908; US20120302910; US20130060110; US20130276785; US20130338459; US20130340751; US20140094670; US20140155704; US20140155713; US20140158132; US20140163897; US20140180026; US20150005591; US20150025494; US20150034082; US20150034084; US20150044305; US20150045686; US20150068526; US20150072023; US20150174158; US20150272988; US20160022731; US20160045154; US20160206839; US20160210747; US20160243330; US20160279165; US20160296719; US20160302704; US20160338597; US20170065631; US20170095630; US20170095631; US20170143758; US20170238841; US20170239289; US20180049654; US20180125883; US20180133246; US20180169320; US20180228398; US20180243526; US20190060595; US20190150789; US20190209795; US20190209796; US20190365281; US20190381264; US20200215102; US20200246573; US20200276229; US20200281965; US20200324071; US20210205565; US20210251520; US20210260317; US20210299370; US20210299379; U.S. Pat. Nos. 5,335,656; 5,677,290; 5,679,649; 5,954,050; 6,004,945; 6,017,315; 6,180,616; 6,342,040; 6,467,477; 6,468,222; 6,544,192; 6,629,934; 6,642,209; 6,745,764; 6,807,965; 6,849,049; 6,899,683; 6,899,684; 6,938,619; 6,955,650; 6,986,347; 7,024,235; 7,034,692; 7,127,278; 7,152,604; 7,201,734; 7,247,154; 7,308,894; 7,565,905; 7,668,579; 7,992,556; 7,997,271; U.S. Pat. Nos. 8,025,052; 8,187,201; 8,666,467; 8,728,001; 8,755,857; 8,862,196; 8,882,703; 8,932,227; 9,042,952; 9,198,586; 9,352,115; 9,521,971; 9,668,661; 9,687,176; 9,962,512; WO1989009565; WO1999020332; WO1999043388; WO1999062403; WO2001008554; WO2001056454; WO2001095971; WO2002071017; WO2003009751; WO2003030979; WO2003057296; WO2003098385; WO2004060443; WO2005065540; WO2007062029; WO2008146264; WO2008148134; WO2010045295; WO2011023961; WO2011029073; WO2012024106; WO2012095764; WO2012142608; WO2012162048; WO2013116389; WO2013132497; WO2013132498; WO2013132499; WO2013132500; WO2013132503; WO2014136111; WO2015106280; WO2015184165; WO2016030760; WO2016094749; WO2016172555; WO2017042636; WO2017059292; WO2017130066; WO2018130673; WO2019090075; WO2019090080; and WO2019156639.

### SUMMARY OF THE INVENTION

The present invention provides a face mask having a colorimetric carbon dioxide sensor visible during use. A colorimetric indicator for carbon dioxide may operate using pH changes due to formation and disassociation of carbonic acid from exhaled carbon dioxide and moisture.

The indicator is preferably in a portion of the mask that is continuously flushed with oxygen, so that between respiratory cycles, the indicator is reset. Practically, the indicator may have an indication half-life of 1-5 seconds, with a saturation response of e.g., 38 mmHg, and sensitivity between <4 mmHg and e.g., 38 mmHg. It is noted that in order to gain higher sensitivity over the range, multiple indicators with different colorimetric responses may be provided, for example one with a saturation of 38 mmHg CO₂, and sensitivity over a range of <4 mmHg to 38 mmHg, and another colorimetric indicator which saturates at 10 mmHg, providing an indication of minimally acceptable ventilation. This permits saturation under normal end tidal conditions of 35-45 mmHg, while remaining sensitive to intermittent apnea and distinguishing between low stable respiratory activity and obstructed or failed breathing. The technology preferably avoids electronic sensors, readouts, or alarms, though in various cases these are acceptable. The technology preferably presents the indicator to be visible by an observer a few feet to a few yards away from different vantage points. This requirement may require that the mask have multiple ports accessible for an indicator, depending on patient orientation, or multiple indicators provided.

According to another embodiment, the mask is available for use before initiation of CPR, to permit rapid determination of breathing status and need for further airway intervention by healthcare professionals. In such cases, no oxygen intake port is required, and the sensor is designed to show color change due to exhaled gas, especially where the patency of the respiratory path is unknown. The same mask may be used during CPR to avoid direct contact. When used with rescue breaths, exhaled carbon dioxide from the administrator will prevent use of the colorimetric indicator.

In one embodiment, a single optimally placed indicator is illuminated (e.g., transilluminated) with a white light emitting diode (LED) or chemiluminescent illuminator glow stick). Alternately, the colorimetric indicator may be provided in the wall of a fiber optic or light pipe, and illumination follow a reflected light path. The color is transmitted through the fiber or light pipe to any one or more destinations. An LED and battery may be provided which lasts 24 hours, assuring continuous operation throughout a procedure. Based on the same illuminator, an alarm may be provided which is triggered by persistence of a color condition for more than 5 seconds. For example, a phototransistor or other photosensor preceded by a color filter, leading to an integrator and a comparator which drives a speaker and/or optical alarm, provide the basic circuit.

In order to properly determine respiratory activity distinguished from abnormal conditions, the exhaled air should flow over the indicator over the full range of flow rates through the mask. Typically, the entry for oxygen is a center port under the nose. At high flow rates, little or no exhaled carbon dioxide will be present at that location. Exhaled air from a respirator mask is typically vented through side ports adjacent to the nose. These side ports may have a baffle or filter. Therefore, a filter formed of indicator material may be provided.

The chemical carbon dioxide detectors may also be encased in housings built in the mask itself, or have the chemical indicator embedded into the wall of the mask.

The mask may have multiple fenestrated sites to provide low resistance exits from the mask to facilitate contact with the chemical CO₂ detector. The patient breathes out carbon dioxide-rich air that flows through the chemical detector, changing its pH, which leads to a visual color change. With each respiration cycle, the carbon dioxide rich gas expelled from the patient dissipates via or in combination with the natural gas laws of concentration dissipation.

Chemical carbon dioxide detectors can use many chemical laws/reactions to provide a visual color change that is rapid and reversible, and changes in the presence or absence of gaseous carbon dioxide. The detectors produce an alternating color change during inspiration and expiration (i.e., the presence or absence of carbon dioxide) due to the pH changes. Oxygen face masks are used to supply a steady flow of oxygen via a plastic tube to a mask entry port to be inhaled by the patient during inspiration.

It is therefore an object to provide a facemask having a colorimetric indicator responsive to carbon dioxide exhaled by a wearer. The colorimetric indicator may protrude from the front of the mask, or be provided as a coating on inner surfaces of the facemask. The indicator preferably has colorimetric calibration markings located at visually proximate locations to facilitate accurate readings of the indicator.

It is also an object to provide a face mask, comprising: a port configured to permit a flow of gas; a shell, configured to surround a mouth and nose of a human face; and a reversible colorimetric carbon dioxide indicator, configured to receive a flow of exhaled air from the human face and to colorimetrically distinguish between respiration-induced flow of carbon dioxide in the exhaled air, and a respiratory obstruction.

The port may be configured to receive the flow of gas, further comprising a second port configured to permit flow of exhaled air from the human face out of the face mask.

It is a further object to provide a face mask, comprising: an inlet port configured to receive a flow of gas; a shell, configured to surround a mouth and nose of a human face, the shell having a plurality of sealed sampling ports; with female luer lock connector and male cap on the body of the mask; an outlet port configured to permit flow of exhaled air from the human face out of the face mask; and a reversible colorimetric carbon dioxide indicator, configured to pierce a respective sealed sampling port, and through the pierced sampling port, receive a flow of exhaled air from the human face and to colorimetrically distinguish between respiration-induced flow of carbon dioxide in the exhaled air, and a respiratory obstruction.

It is a further object to provide a method of assessing breathing through a face mask configured to surround a mouth and nose of a human face, comprising: receiving a flow of a breathable gas into an inlet port of the faced mask; receiving a flow of exhaled air from a patient wearing the face mark; and interacting the flow of exhaled air with a reversible colorimetric carbon dioxide indicator, to colorimetrically distinguish between respiration-induced flow of carbon dioxide in the exhaled air and a respiratory obstruction.

It is another object to provide a face mask, comprising: an inlet port configured to receive a inflow of gas through an inlet check valve; a shell, configured to surround a mouth and nose of a human face, the shell having a plurality of sealed sampling ports; an outlet port configured to permit flow of exhaled air from the human face out of the face mask, e.g., through an outlet check valve which may be provided separately from the face mask; and a reversible colorimetric carbon dioxide indicator, configured to pierce a respective sealed sampling port, and through the pierced sampling port, receive a flow of exhaled air from the human face and to colorimetrically distinguish between respiration-induced flow of carbon dioxide in the exhaled air, and a respiratory obstruction.

The reversible colorimetric carbon dioxide indicator may comprise a disk, a ring, or a coating on an inner surface of the face mask. The colorimetric indicator may be non-planar, e.g., conical, spherical (concave or convex surface), etc.

The reversible colorimetric carbon dioxide indicator may be responsive to exhaled carbon dioxide over a range of inlet port flow of oxygen over a range of 0.5-5, 7, 10, 12, 15, or 20 liters per minute.

The shell may comprise clear plastic, further comprising an adjustable nose piece; with female luer lock connector and male cap on the body of the mask; and an adjustable head strap.

The reversible colorimetric carbon dioxide indicator may be attachable to and detachable (removable) from the face mask.

The face mask may further comprise an illuminator configured to illuminate the reversible colorimetric carbon dioxide indicator and to project colored light from the reversible colorimetric carbon dioxide indicator. A light pipe may transmit the projected colored light.

The method may further comprise attaching the reversible colorimetric carbon dioxide indicator to the mask and/or removing it from the face mask.

The method may further comprise illuminating the reversible colorimetric carbon dioxide indicator, and projecting colored light from the reversible colorimetric carbon dioxide indicator.

### BRIEF DESCRIPTION OF THE DRAWINGS

FIG. 1 shows a front view of a first embodiment of the invention.

FIG. 2 shows a side perspective exploded view of the first embodiment of the invention.

FIG. 3 shows a side perspective exploded view of a second embodiment of the invention.

FIG. 4 shows a front view of a third embodiment of the invention.

FIG. 5 shows a side perspective exploded view of the third embodiment of the invention.

FIG. 6 shows front view of a modification of FIG. 4 having a port above the nose.

FIG. 7 shows side perspective exploded view of a modification of FIG. 5 having a port above the nose.

FIG. 8 shows a front view of a fourth embodiment of the invention.

FIG. 9 shows a front exploded view of the fourth embodiment of the invention.

FIG. 10 shows a side perspective view of the fourth embodiment of the invention.

FIG. 11 shows a front view of a fifth embodiment of the invention.

FIG. 12 shows a side perspective view of the fifth embodiment of the invention.

FIG. 13 shows a side perspective view of a sixth embodiment of the invention.

FIG. 14 shows a rear perspective exploded view of the sixth embodiment of the invention.

FIG. 15 shows a rear view of a seventh embodiment of the invention.

FIG. 16 shows a front view of a modification of FIG. 15 having a port located above the nose.

FIG. 17 shows a side perspective exploded view of a modification of FIG. 13 having a port located above the nose.

FIG. 18 shows a side perspective exploded view of the seventh embodiment of the invention.

FIG. 19 shows a rear perspective exploded view of the seventh embodiment of the invention.

FIG. 20 shows a rear view of the seventh embodiment of the invention.

FIG. 21 shows a rear perspective exploded view of a eighth embodiment of the invention.

FIG. 22 shows a side perspective exploded view of the eighth embodiment of the invention.

FIG. 23 shows a front view of the eighth embodiment of the invention.

FIG. 24 shows a schematic view of a rectangular colorimetric indicator.

FIG. 25 shows a schematic view of a circular colorimetric indicator.

FIG. 26 shows a prior art Nellcor EZCap colorimetric indicator.

FIG. 27 shows a capnogram.

### DETAILED DESCRIPTION OF THE INVENTION

A preferred embodiment of the invention provides an oxygen mask with incorporated carbon dioxide indicators. Carbon dioxide, which is a normal component of exhaled gas, produces a changed visual display showing its presence via color change. An observer may then observe an alternating color change during inspiration and expiration (i.e., the presence or absence of carbon dioxide) due to the pH changes of the detecting material incorporated in the mask when mixed with carbon dioxide.

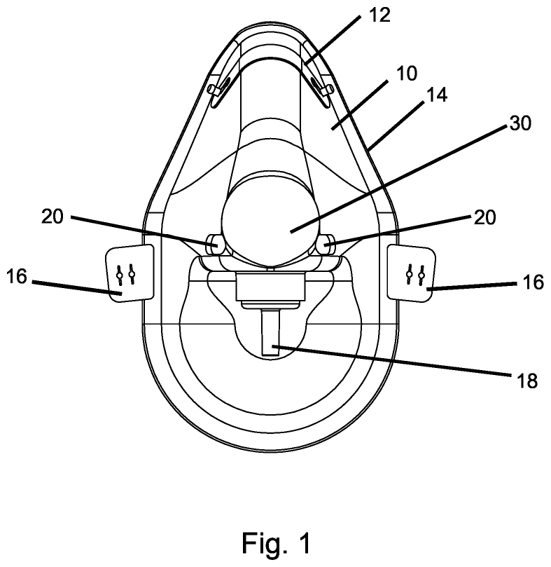

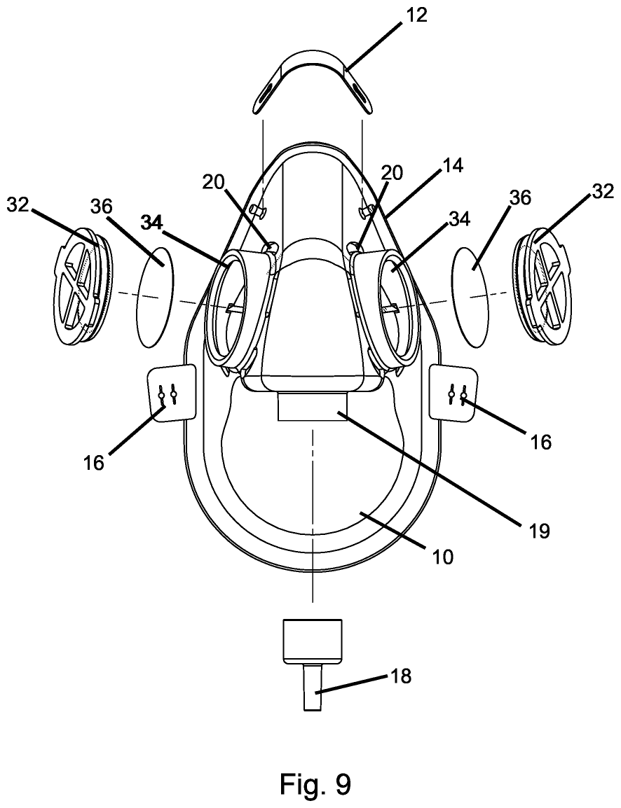

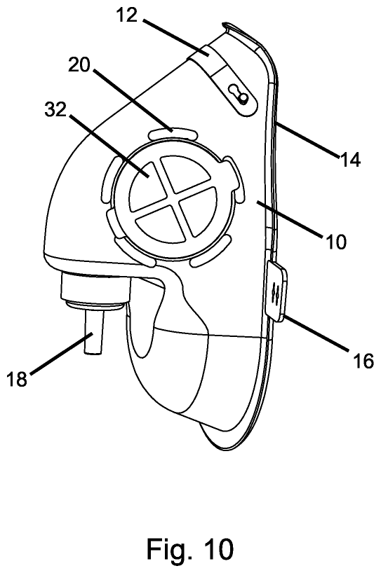

FIGS. 1-12 show a so-called simple facemask design, which does not seal around the edge.

In a facemask, oxygen is supplied through a hose adapter 18, which connects to inlet 19 below the nose portion of the mask 10. The mask 10 and its major components are formed of a transparent flexible plastic, such as polyvinyl chloride. The inlet 19 is located midline, below the nose of the mask 10 and above the lower border of the edge 14 of the mask 10.

The oxygen is inhaled by the individual during inspiration. The pH indicator of the carbon dioxide detector 30 is placed near fenestrated ports 20 for passing exhaled breath out of the mask 10. The fenestrated ports 20 can be placed on one, both sides, front, or the dome of the mask, for example.

The mask 10 is held to the patient's face by a set of adjustable elastic straps (not shown) affixed to strap holders 14, with female luer lock connector 40 and male cap 42 on the body of the mask 10. The nose bridge of the mask 10 is adjustable to fit by a plastically-deformable element 12 which is, for example, a soft aluminum sheet.

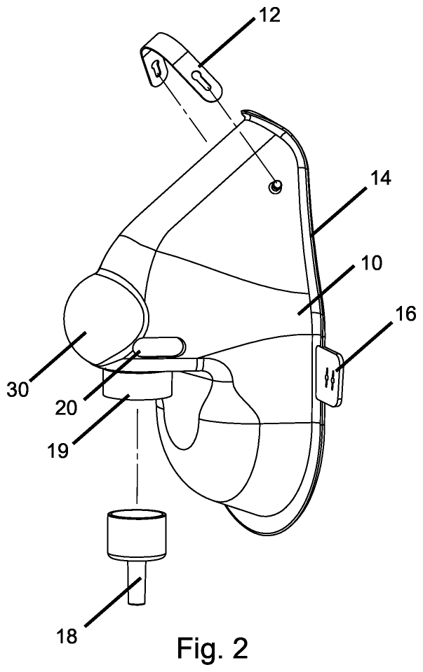

As shown in FIGS. 1 and 2 of the first embodiment of the invention, the carbon dioxide detector 30 is provided as a bulb in front of the nose, which is visible >180 degrees in front of the patient. The inside of the bulb 30 is coated with indicator dye. The fenestrated ports 20 are located such that an exhale flow pattern distributes gas within the bulb 30, while between breaths the bulb 30 is flushed with clean oxygen from the hose adapter 18.

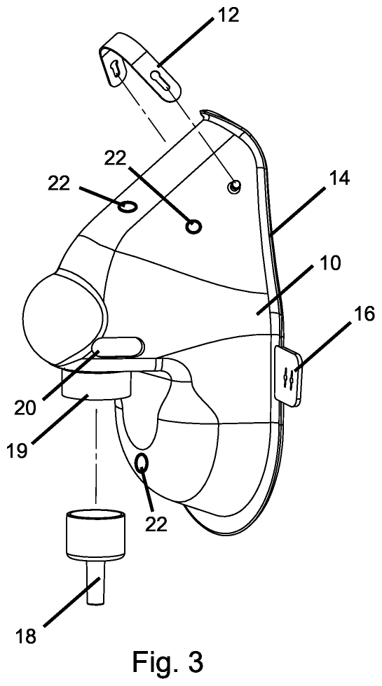

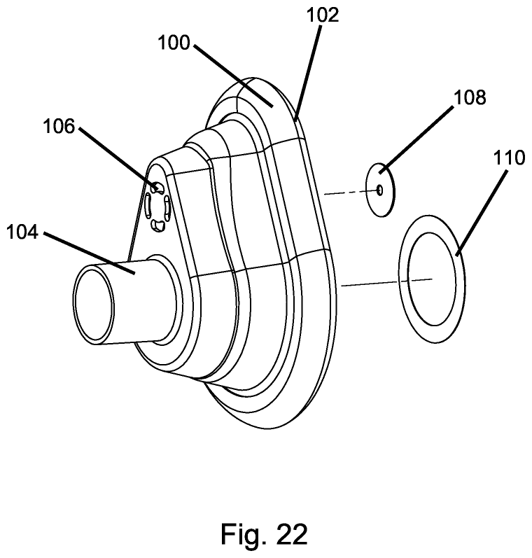

In a second embodiment shown in FIG. 3, is similar to the first embodiment, but the mask 10 has a series of ports 22, which are sealed with a membrane, in various portions of the mask 10. A colorimetric capnometer, such as the Nellcor EZCap™ II, shown representatively in FIG. 22, may be inserted into one or more ports 22, piercing the membrane to allow a flow path from inside the mask to the outside. This allows a caregiver to select the desired location or locations for the indicator based on the orientation of the patient. The orientation of the colorimetric capnometer may be adjusted for visibility.

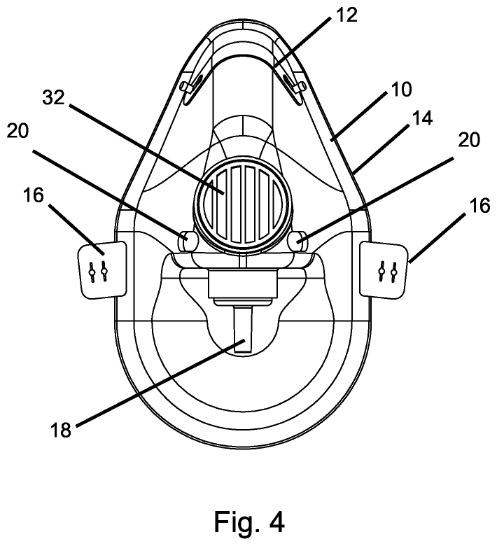

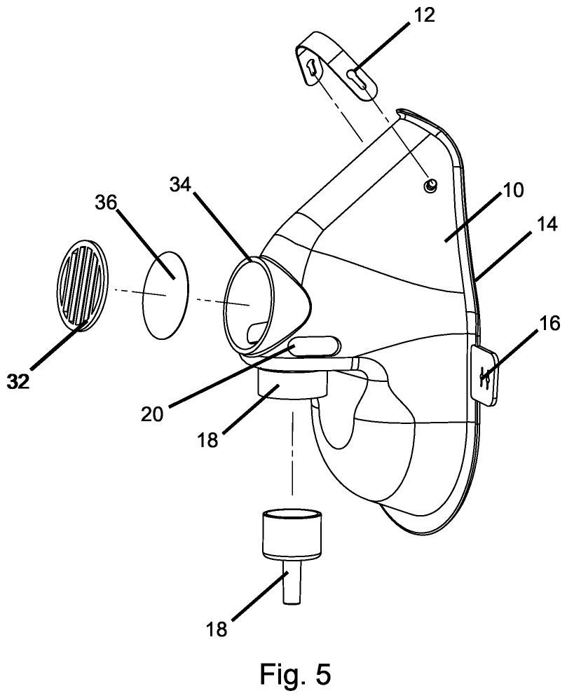

As shown in FIGS. 4 and 5 of the third embodiment of the invention, the carbon dioxide detector 36 medium is a film or paper which is held behind a grill 32, in front of a shell 34. The fenestrated ports 20 are located such that an exhale flow pattern distributes gas around the carbon dioxide detector 36 medium, while between breaths the carbon dioxide detector 36 medium is flushed with clean oxygen from the hose adapter 18.

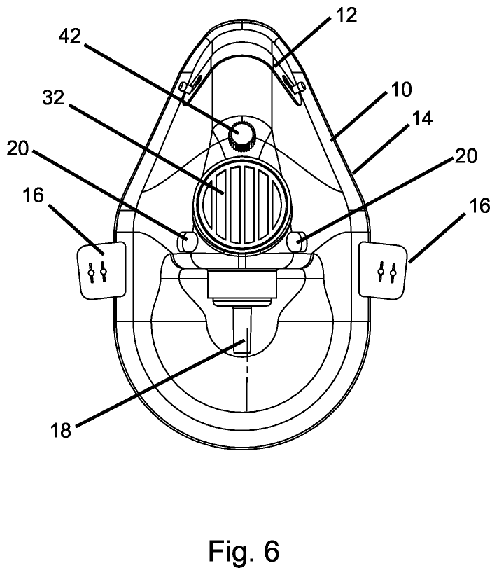

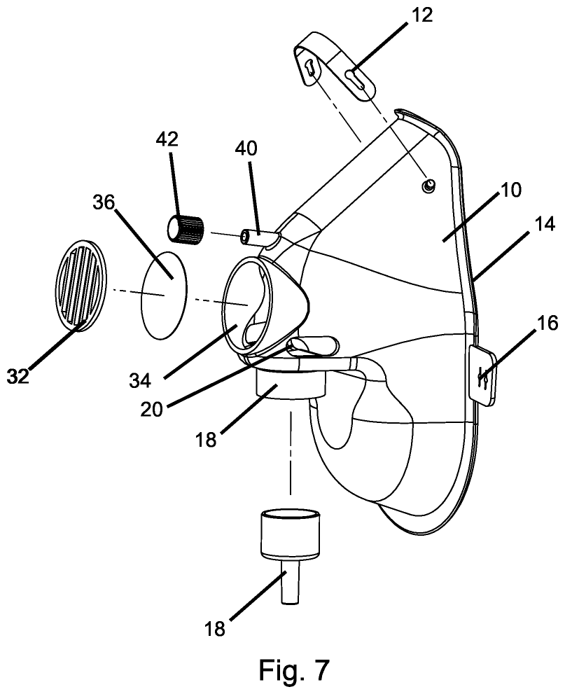

FIGS. 6 and 7 show modifications of FIGS. 4 and 5, respectively, having a port 40 above the nose. The port 40 may provide a vent for exhaled air, an interface to a sampling tube, or a separate colorimetric indicator (not shown). When not in use, a cap 42 blocks gas flow.

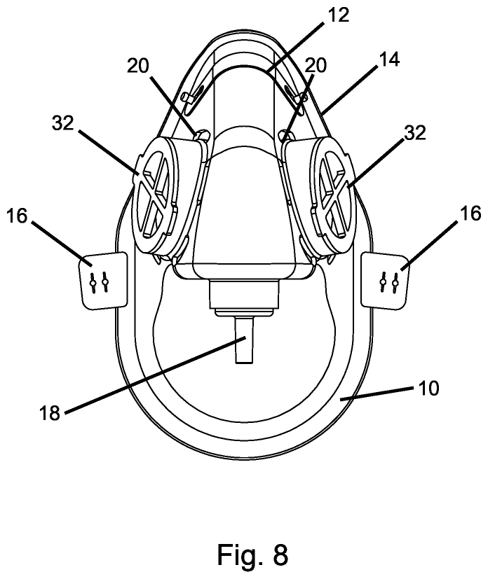

As shown in FIGS. 8, 9 and 10 of the fourth embodiment of the invention, a pair of indicators 36 are provided on the left and right sides of the mask 10, behind grills 32 and in front of shells 34. The fenestrated ports 20 are located surrounding the carbon dioxide detector 36 medium, and an exhale flow pattern distributes gas around the carbon dioxide detector 36 medium venting through the fenestrated ports 20, while between breaths the carbon dioxide detector 36 medium is flushed with clean oxygen from the hose adapter 18.

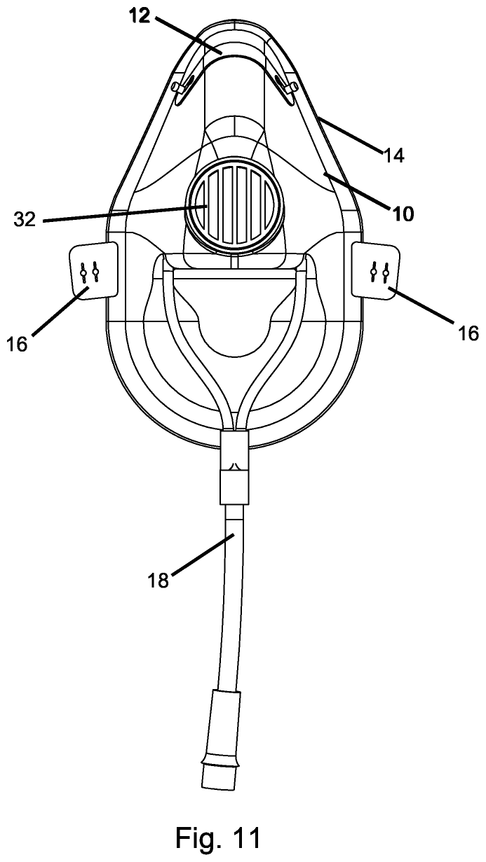

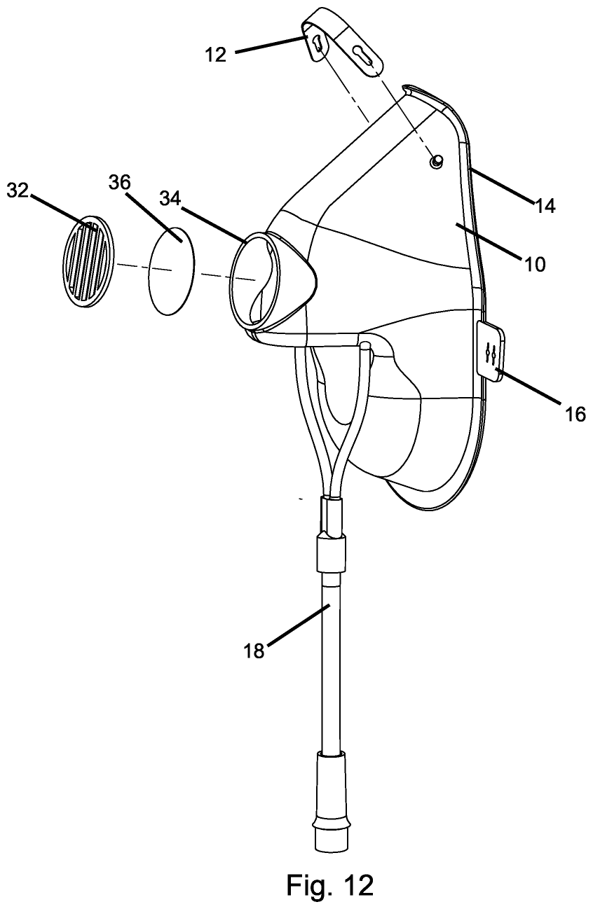

FIGS. 11 and 12 of the fifth embodiment of the invention has a centrally-located the carbon dioxide detector 36 medium indicator, behind grill 32 and in front of shell 34. The exhale flow path (not shown) is through ports below the nostrils. Oxygen is suppled through a hose adapter 18, which in this case splits into two hoses feeding the left and rights sides of the mask. This configuration supports high flow rates, and the dual tubes are more flexible than a single tune with the same cross section area and flow capacity. In a variant of the fourth embodiment, the hose adapter has dual lumens, and one of the tubes leading to the mask is used as a sampling port for exhaled gas. Typically, only a small portion of the exhaled gas passes through the sampling port, and the majority is released to the atmosphere.

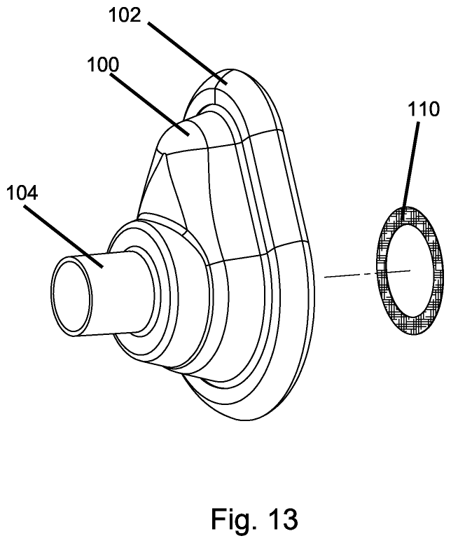

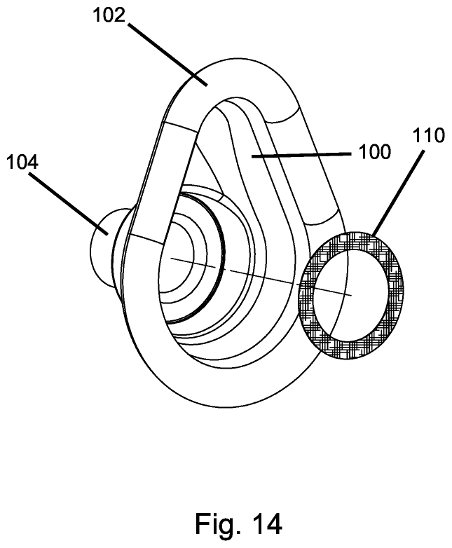

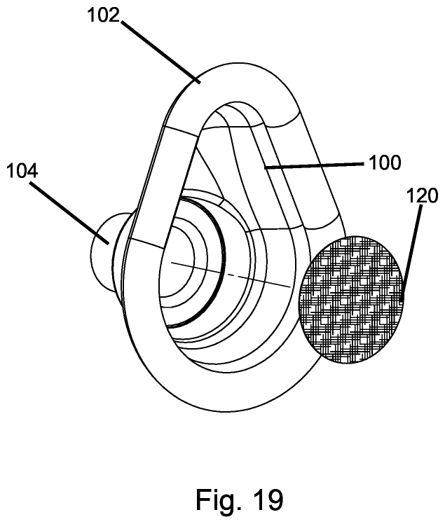

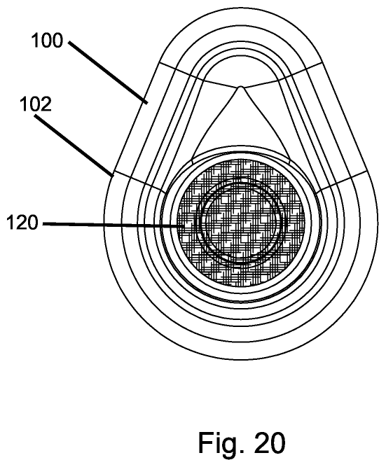

FIGS. 13-20 show a bag valve mask (positive pressure mask) that has a seal around the face.

FIGS. 13, 14 and 15 of the sixth embodiment of the invention show a mask 100 in which oxygen or air is fed in front of the nose through port 104. The mask has a gas-filled edge 102 that provides a compliant seal against the face of the wearer. The indicator 110 is configured as a ring around the inlet port 104, visible through the mask from 180 degrees. This design is suitable for administering cardiopulmonary resuscitation (CPR), with the caregiver isolated from the patient. Note that the carbon dioxide detector medium indicator 120 will not measure the patient's carbon dioxide exhalation accurately during CPR, and the indicator is provided for an initial assessment of patient breathing before commencement of CPR, and perhaps periodically after commencement. No exhale ports need be provided because the mask is readily removed from the face between breaths.

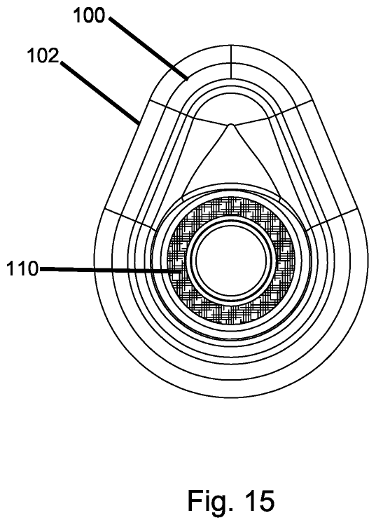

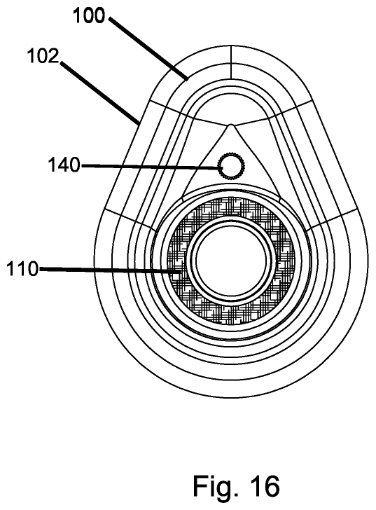

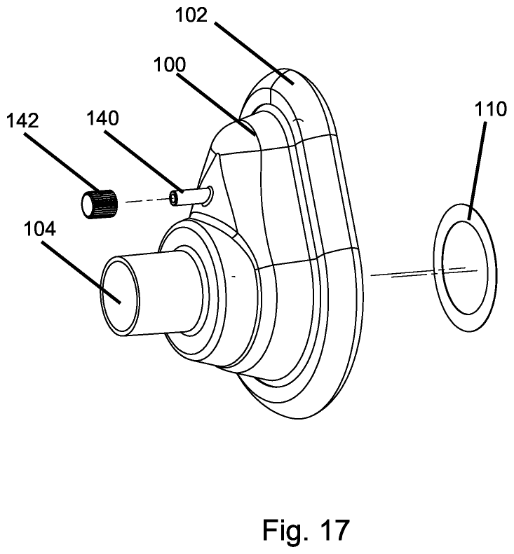

FIGS. 16 and 17 show a modification of FIGS. 15 and 13, respectively, having a port 140 located above the nose. The port 140 may provide a vent for exhaled air, an interface to a sampling tube, or a separate colorimetric indicator (not shown). When not in use, a cap 142 blocks gas flow.

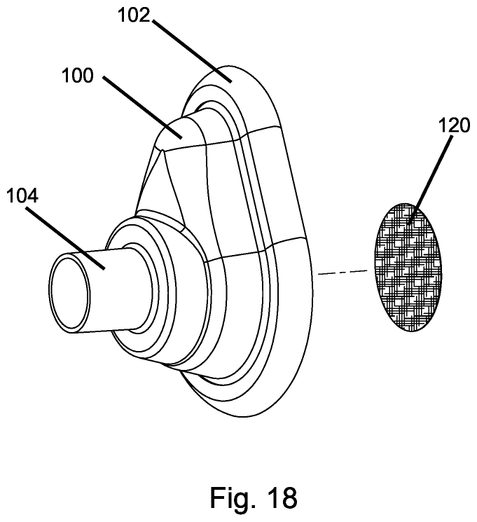

FIGS. 18, 19 and 20 of the seventh embodiment of the invention show a mask 100 in which oxygen or air is fed in front of the nose through port 104. The mask has a gas-filled edge 102 that provides a compliant seal against the face of the wearer. The indicator 120 is configured as a disk around the inlet port 104, visible through the mask from front and sides. In this case, the indicator 120 disk may be porous, and allow air flow through it during use, or mounted as a baffle for air flow around the disk to ensure contact of the flowing air with the surfaces of the disk. As with the sixth embodiment, the design of the seventh embodiment is suitable for administering cardiopulmonary resuscitation (CPR), with the caregiver isolated from the patient, in this case with a physical barrier than can serve as a filter. The carbon dioxide detector medium indicator 120 can measure the caregiver's exhaled carbon dioxide during CPR, and help assure breaths provided to the patient are not stale.

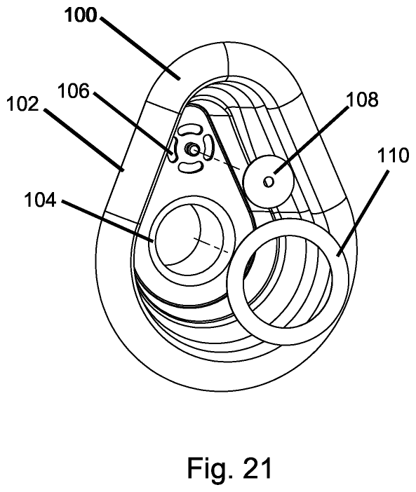

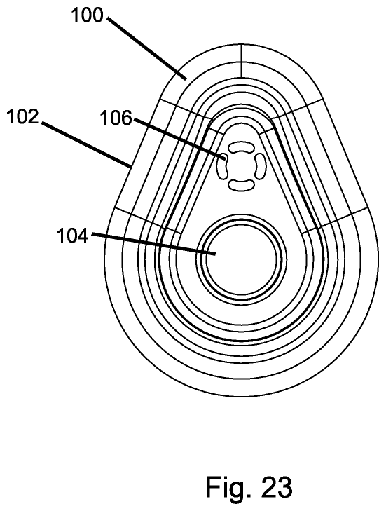

FIGS. 21-23 show a barrier mask to permit a caregiver to administer rescue breaths to a patient without physical contact. A caregiver breathes through a tube which has a one-way inlet check valve to prevent backflow from the patient to the caregiver. An outlet check valve in the mask permits inhalation by the patient when the mask is sealed against the face of the patient.

FIGS. 21, 22 and 23 of the eighth embodiment are also similar to the sixth embodiment, employing a mask 100 in which oxygen or air is fed in front of the nose through port 104. The mask has a gas-filled edge 102 that provides a compliant seal against the face of the wearer. The indicator 110 is configured as a ring around the inlet port 104, visible through the mask from 180 degrees. This design is suitable for administering cardiopulmonary resuscitation (CPR), with the caregiver isolated from the patient. A valved inhale port 106 is provided with an elastomeric sealing disk 108 mounted on a central pin. In this configuration, if a low oxygen flow rate is provided through the port 104, any excess air is drawn through the valved inhale port 106. For example, if a patient is provided with 2 liters per minute of oxygen, at least 50% of the inhaled gas will bypass through the valved inhale port 106. During exhale, the patient's breath will pass near the ring indicator 110, and either back through the inlet port 104 if possible, or around the edge 102 of the mask 100.

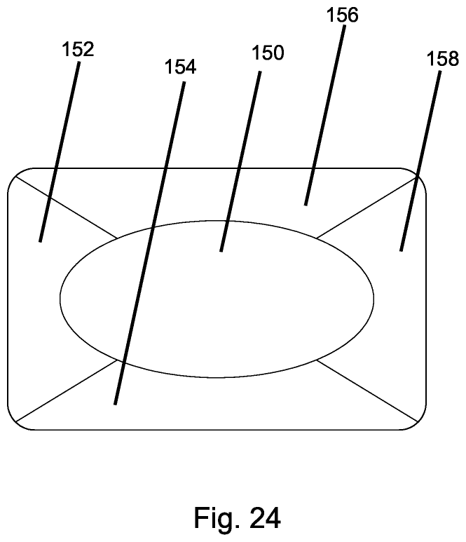

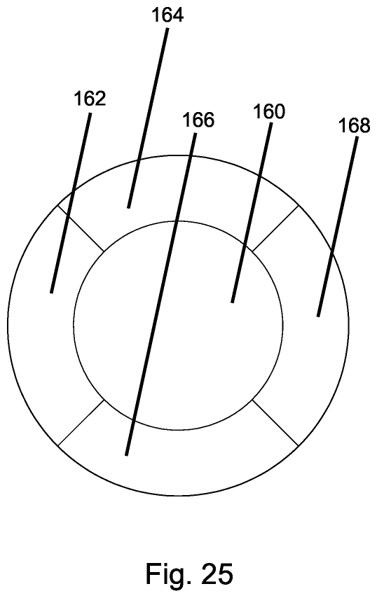

FIGS. 24 and 25 show rectangular 150 and circular 160 colorimetric indicators, with surrounding regions 152, 154, 156, 158, 162, 164, 166, 186 on which accurate colors are printed showing the color of the indicator under respective conditions of CO₂, e.g., 0.03, 0.5, 2.0, and 5.

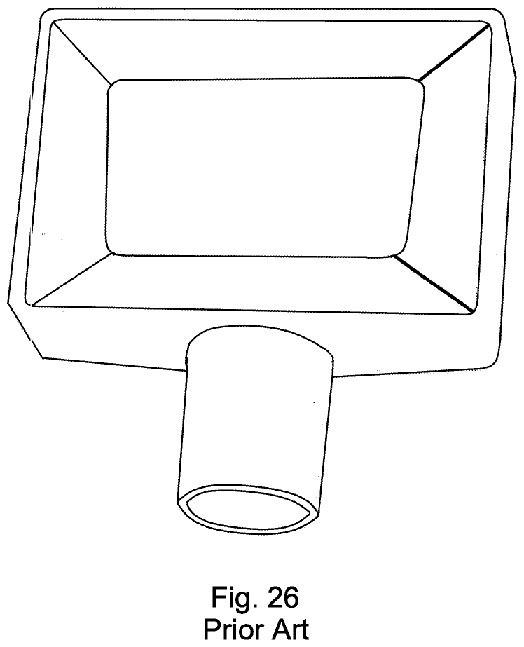

FIG. 26 shows a perspective view of a prior art Nellcor EZCap™ II colorimetric indicator capnometer. Such devices are known for use with endotracheal tubes, and have not been integrated with or disposed on respirator masks.

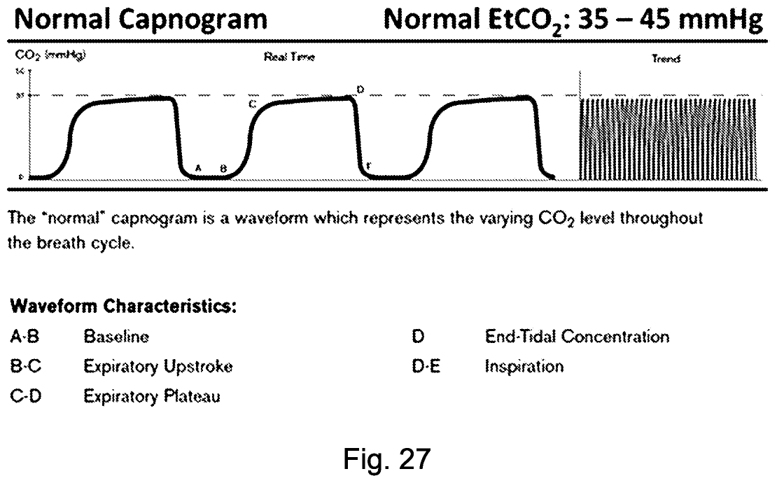

FIG. 27 shows a normal capnogram, showing changes of carbon dioxide in exhaled breath over time. Clean air has a trace amount of carbon dioxide, while the end tidal CO₂ is about 25-45 mmHg (4.6%-6%).

The carbon dioxide detectors may use various chemistries, e.g., metacresol purple, to provide a visual color change (purple in air, yellow in 4%+CO₂) that is rapid and reversible, and changes in the presence or absence of gaseous carbon dioxide. The detectors may be encased in a housing that allows for optimal visualization from a wide range of vantage points, regardless of the orientation of the mask to the observer. The detector housings isolate the chemical agents from the patient and room environment. These housings allow for easy attachment to and removal from the masks various attachment points, as well as an optimal air flow to allow for the largest detection of carbon dioxide and therefore largest color change in the respiratory cycle of the patient.

The invention alleviates the need for subjective or unreliable measures of breathing, such as chest movement or the expensive, computerized, battery operated capnometers. The colorimetric indicator is preferably single-use, and may produce valid results for a duration.

It should be understood that the preferred embodiments and examples described herein are for illustrative purposes only and are not to be construed as limiting the scope of the present invention, which is properly delineated only in the appended claims.

1. A face mask, comprising:
a port configured to permit a flow of gas;
a shell, configured to surround a mouth and nose of a human face; and
a reversible colorimetric carbon dioxide indicator, configured to receive a flow of exhaled air from the human face and to colorimetrically distinguish between respiration-induced flow of carbon dioxide in the exhaled air, and a respiratory obstruction.

2. The face mask according to claim 1, wherein the port is configured to receive the flow of gas, further comprising a second port configured to permit flow of exhaled air from the human face out of the face mask.

3. The face mask according to claim 1, wherein the reversible colorimetric carbon dioxide indicator comprises a ring.

4. The face mask according to claim 1, wherein the reversible colorimetric carbon dioxide indicator comprises a coating on an inner surface of the face mask.

5. The face mask according to claim 1, wherein the reversible colorimetric carbon dioxide indicator is responsive to exhaled carbon dioxide over a range of inlet port flow of oxygen over a range of 0.5-15 liters per minute.

6. The face mask according to claim 1, wherein the shell comprises clear plastic, further comprising an adjustable nose piece; and an adjustable head strap.

7. The face mask according to claim 1, wherein the reversible colorimetric carbon dioxide indicator is attachable to the face mask.

8. The face mask according to claim 1, wherein the reversible colorimetric carbon dioxide indicator is removable from the face mask.

9. The face mask according to claim 1, further comprising an illuminator configured to illuminate the reversible colorimetric carbon dioxide indicator and to project colored light from the reversible colorimetric carbon dioxide indicator.

10. A method of assessing breathing through a face mask configured to surround a mouth and nose of a human face, comprising:
receiving a flow of a breathable gas into an inlet port of the faced mask;
receiving a flow of exhaled air from a patient wearing the face mark; and
interacting the flow of exhaled air with a reversible colorimetric carbon dioxide indicator, to colorimetrically distinguish between respiration-induced flow of carbon dioxide in the exhaled air and a respiratory obstruction.

11. The method according to claim 10, wherein the reversible colorimetric carbon dioxide indicator comprises a non-planar structure.

12. The method according to claim 10, wherein the reversible colorimetric carbon dioxide indicator comprises a ring.

13. The method according to claim 10, wherein the reversible colorimetric carbon dioxide indicator comprises a coating on an inner surface of the face mask.

14. The method according to claim 10, wherein the reversible colorimetric carbon dioxide indicator is responsive to exhaled carbon dioxide over a range of inlet port flow of oxygen over a range of 0.5-15 liters per minute.

15. The method according to claim 10, wherein the shell comprises clear plastic, further comprising an adjustable nose piece; and an adjustable head strap.

16. The method according to claim 10, further comprising attaching the reversible colorimetric carbon dioxide indicator to the face mask.

17. The method according to claim 10, further comprising removing the reversible colorimetric carbon dioxide indicator from the face mask.

18. The method according to claim 10, further comprising illuminating the reversible colorimetric carbon dioxide indicator, and projecting colored light from the reversible colorimetric carbon dioxide indicator.

19. A face mask, comprising:
an inlet port configured to receive an inflow of gas through an inlet check valve;
a shell, configured to surround a mouth and nose of a human face, the shell having a plurality of sealed sampling ports;
an outlet port configured to permit flow of exhaled air from the human face out of the face mask; and
a reversible colorimetric carbon dioxide indicator, configured to pierce a respective sealed sampling port, and through the pierced sampling port, receive a flow of exhaled air from the human face and to colorimetrically distinguish between respiration-induced flow of carbon dioxide in the exhaled air, and a respiratory obstruction.

20. The face mark according to claim 19, wherein the reversible colorimetric carbon dioxide indicator is responsive to exhaled carbon dioxide over a range of inlet port flow of oxygen over a range of 0.5-15 liters per minute.
